(19) United States
(12) Patent Application Publication
(10) Pub. No.: US 2004/0073175 A1
(43) Pub. Date: Apr. 15, 2004

(54) Infusion system

(76) Inventors: James D. Jacobson, Lindenhurst, IL (US); Tuan Bui, Green Oaks, IL (US); Qui Chau, Skokie, IL (US); Francis C. Kowalik, Deerfield, IL (US); Alice Jandrisits, Des Plaines, IL (US); Biren Lal, Lake Zurich, IL (US); Mark Williamson, Wonder Lake, IL (US)

Correspondence Address: Francis C. Kowalik, Esq., Corporate Counsel, Law Department, BAXTER INTERNATIONAL INC., One Baxter Parkway, DF3-2E, Deerfield, IL, 60015, US

(21) Appl. No.: 10/643,259

(22) Filed: Aug. 19, 2003

Related U.S. Application Data
(63) Continuation-in-part of application No. 10/040,887, filed on Jan. 7, 2002.

(51) Int. Cl. A61M 5/00

(52) U.S. Cl. 604/251; 604/411

(57) ABSTRACT

An infusion system is disclosed for delivering fluid wherein the system has a flow control element within a spike member (100). The spike member (100) is a fluid extraction spike member having a fluid passageway (110) and a micro-electromechanical system (MEMS) element (108) operatively connected to the fluid extraction spike member (100).

### RELATED APPLICATIONS

The present application is a continuation-in-part of U.S. application Ser. No. 10/040,887 filed on Jan. 7, 2002 and entitled, “Infusion System,” which application is incorporated by reference herein and made a part hereof, and upon which a claim of priority is based.

### TECHNICAL FIELD

The present invention generally relates to a medical fluid flow control system such as an infusion system, and more particularly to a method and apparatus for control of such systems using a micro-electromechanical element. The present invention also generally relates to a medical infusion set having an active pump element within a spike, and more particularly, to a disposable medical infusion set with a spike having a micro-electromechanical system (MEMS) element, such as a MEMS pump, incorporated therein.

### BACKGROUND OF THE INVENTION

Generally, medical patients require precise delivery of either continuous medication or medication at set periodic intervals. Medical fluid flow control systems that include medical pumps have been developed to provide controlled drug infusion. Using the pump, the drug can be administered at a precise rate that keeps the drug concentration within the therapeutic margin and out of a possible toxic range with certain drugs. These sophisticated medical pumps provide appropriate drug delivery to the patient at a controllable rate that does not require frequent medical attention.

These pumps are often part of an infusion system that is typically used to deliver medication to a patient. Infusion pumps are generally used when the accuracy available via gravity-based infusion is unacceptable or undesirable. In the case of chronic pain, an infusion system is used when oral or topical medications fail to provide effective pain relief or cause uncomfortable side effects. An infusion system may also be used when delivering medication to a specific site or organ proves to be more effective or to cause fewer uncomfortable side effects than delivering the medication systematically to the entire body. The use of an infusion system allows a physician to target sites within the body for more effective delivery of a medication. The infusion system can deliver medication to a patient at a controlled rate as prescribed by a physician.

A medical fluid flow control system can be an infusion system wherein a medication is delivered to a patient, or a draw-type system wherein a fluid is taken from a patient and delivered to a separate container. The system typically includes several different components including tubing, a pump, a reservoir, a spike and an access port. The system could also have other components such as valves and sensors. The components of the system must remain sterile. Some components such as the tubing, container, spike and access port are typically disposable. Other components may be durable or reusable elements such as the pump, valves and any required electronic controllers or power supplies. These components are typically larger, expensive pieces of equipment traditionally packaged into a single durable or reusable system. In some cases, these components may need to be sterilized, or at least cleaned, prior to their next use. This can be an expensive and time-consuming process. Furthermore, as the pump is often the most costly reusable element of the system, there is increased pressure to use a pump that is less costly and smaller in size, but that can still deliver a medication in a controlled, accurate, and safe manner.

Because infusion pumps are relatively large, the use of multiple infusion channels is cumbersome and thereby limits the ambulation of patients. As infusion pumps can be expensive, bulky, and troublesome with respect to storage, maintenance, and usage, there is a need for improvement in the field of medication delivery.

In order to limit the amount of equipment that requires sterilization, it is desirable to have a medical fluid flow control system that uses as many disposable elements as possible. These components are typically less expensive. Such a system also reduces maintenance concerns.

The present invention is provided to solve these and other problems.

### SUMMARY OF THE INVENTION

The present invention is generally directed to a medical fluid control system such as an infusion system. Medical infusion systems typically include durable or reusable elements, and disposable elements that operate complementarily to provide medication to a patient. Typically, the disposable element is a piece of medical tubing or a customized cassette that is manipulated by a “hardware” system to provide the desired medication delivery. The use of micro-electromechanical systems (MEMS) in the infusion system provides an opportunity to add disposable elements to the infusion system that provide additional functionality. The transfer of certain mechanical features from the durable elements of the infusion system to the disposable elements, permits cheaper construction of the durable elements and provides longer term reliability since the durable elements would not be required to provide the mechanical functions of, for example, pumping and flow control.

According to a first aspect of the invention, the system preferably includes a length of tube and a MEMS element operably connected to the tube. In one preferred embodiment, the element is a MEMS pump. The system can be disposable and implemented with a reusable controller and power source. Other additional elements that may be included in the system are flow valves, flow sensors, and pressure sensors.

According to another aspect of the present invention, a wireless controller is provided to control the MEMS element. The controller may control the element from a remote location.

According to another aspect of the present invention, the system includes a spike member having a passageway for fluids and one or more integral MEMS elements housed within the spike member. In one preferred embodiment, the spike member is a stand-alone, disposable, fluid extraction spike member. The MEMS elements may be, for example, a MEMS pump, valve, flow sensor, pressure sensor, or some combination thereof. The spike can be used in conjunction with other elements of a medical line-set to pump fluids from a rigid or flexible container or reservoir. In the case of a rigid container, the spike can be configured to either force fluid out of the container by pumping air into it or, draw fluid from the container allowing air to be ventilated into the container.

By including MEMS elements within a spike, an excellent packaging approach is possible, allowing a so-called “smartspike” set that includes a spike with an active pump or other elements within it, as well as tubing and access connection. Insertion of the spike into a bag, container or reservoir provides a complete, closed infusion system that may be discarded after use.

Other advantages and features of the present invention will be apparent from the following description of the embodiments illustrated in the accompanying drawings.

### BRIEF DESCRIPTION OF DRAWINGS

FIG. 1 is a schematic diagram of an embodiment of a medical fluid flow control system where a micro-electromechanical system (MEMS) element is connected to a line-set;

FIG. 2 is a schematic diagram of another embodiment of the medical fluid flow control system where a MEMS element and other components including a controller are connected to a line-set in another configuration;

FIG. 3 is a schematic diagram of another embodiment of the medical fluid flow control system where a power source is connected to the line-set and is operably connected to a MEMS pump;

FIG. 4 is a schematic diagram of another embodiment of the medical fluid flow control system where MEMS element communication with the controller is wireless;

FIG. 5 is a schematic diagram of another embodiment of a medical fluid flow control system where the system can be implanted in a body;

FIG. 6 is a schematic view of a medical spike for a medical infusion set or system in accordance with another embodiment of the present invention;

FIG. 7 is a schematic view of a medical spike inserted into a container with associated components of an infusion system;

FIG. 8 is a schematic view of an alternative embodiment of a medical spike inserted into a container with associated components of an infusion system;

FIG. 9 is a schematic view of yet another alternative embodiment of a medical spike inserted in a container with associated components of an infusion system;

FIG. 10 is a schematic view of an embodiment showing an air pump housed within a medical spike; and,

FIG. 11 is a schematic view of an embodiment showing a fluid pump housed within a medical spike.

### DETAILED DESCRIPTION

While this invention is susceptible of embodiments in many different forms, there is shown in the drawings and will herein be described, in detail, preferred embodiments of the invention. The present disclosure is to be considered as an exemplification of the principles of the invention and is not intended to limit the broad aspect of the invention to the embodiments illustrated.

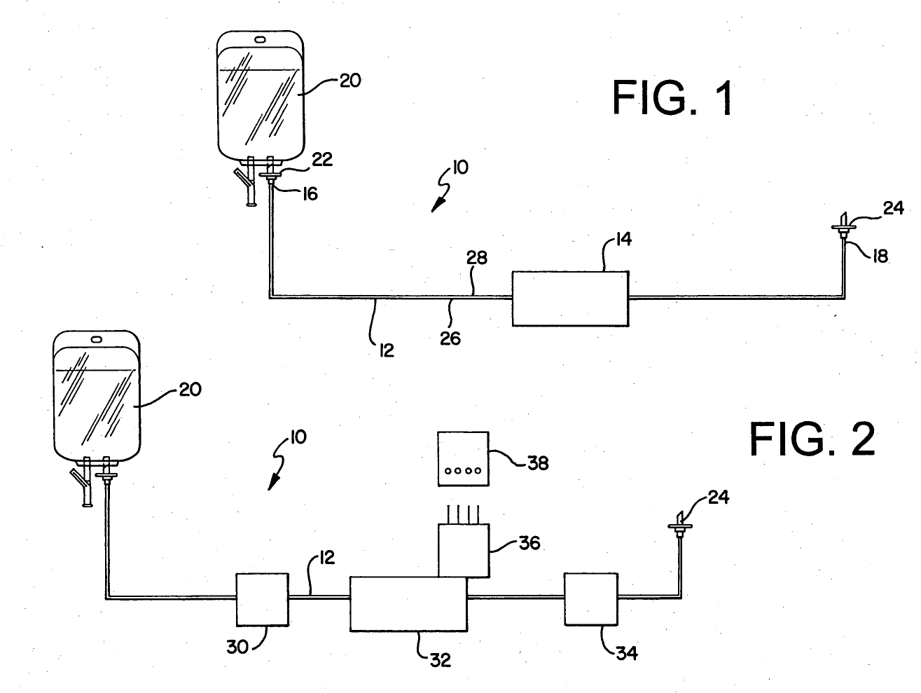

Referring to the drawings, FIG. 1 discloses a medical fluid flow control system of the present invention, generally referred to with the reference numeral 10. The medical fluid flow control system 10 can be configured as an infusion system wherein, for example, a liquid medication is delivered by the system 10 to a patient. It is understood, however, that the system 10 can also be configured as a draw system wherein fluid is taken from a patient and delivered to a container. The medical fluid flow control system 10, in one preferred embodiment, may be in the form of a line-set. The line-set is preferably designed for single use only, disposable after use by patients. The system 10 generally includes a section of tubing 12 and a micro-electromechanical system (MEMS) element 14.

The tubing 12 has a first end 16 and a second end 18. The first end 16 of the tubing 12 is adapted to be connected to a fluid source (a first component) such as an IV bag 20 or other type of reservoir or container. The first end 16 may have a separate connector 22 to connect to the bag 20. The second end 18 of the tubing 12 is adapted to be in communication with, for example, a patient. To that end, the second end 18 may be equipped with an access device 24. The access device 24 can be in the form of a connector for attachment to, for example, a cannula, catheter, syringe, IV line, or any of several other known medical instruments or devices (a second component). The tubing 12 has a generally cylindrical wall 26 defining an interior passageway therethrough 28.

The tubing 12 can be of any suitable medical grade tubing used for procedures requiring a transfer of fluid from at least one source site to at least one recipient site. Exemplary tubing is described in U.S. patent application Ser. No. 08/642,278, entitled “Method of Using Medical Tubings in Fluid Administration Sets,” and U.S. Pat. No. 6,129,876, entitled “Heat Setting of Medical Tubing,” each filed on May 3, 1996, and assigned to the Assignee of this application. Each of these documents is hereby incorporated by reference.

As further shown in FIG. 1, the micro-electromechanical system (MEMS) element 14 is connected to the tube 12. MEMS is a technology that allows for the economical production of tiny electromechanical devices, which can be less than a millimeter in size. MEMS elements are typically fabricated from glass wafers, silicon, or even plastics as the technology has grown far beyond its origins in the semiconductor industry. Each device is an integrated micro-system on a chip that can incorporate moving mechanical parts in addition to optical, fluidic, electrical, chemical and biomedical elements. The resulting MEMS elements are responsive to many types of input, including pressure, vibration, chemical, light, and acceleration. These devices are smaller than conventional machines used for sensing, communication and actuation. As a result, it is possible to use them in places where mechanical devices could not be traditionally used. The batch fabrication techniques associated with MEMS also provide the opportunity to create disposable devices in a cost effective manner. In sum, MEMS elements are not only small in size, but can be economically produced.

The MEMS element 14 can be a number of different components including various types of pumps, a flow valve, a flow sensor, tubing, a pressure sensor or combinations of elements. Because of the actual size of the MEMS element 14, it is understood that the MEMS element 14 is shown schematically in the figures. The MEMS element 14 may be powered by a battery, power supply, or other source of power if necessary. The embodiment shown in FIG. 1 has the source of power and controller as part of the MEMS element 14. As described below, the power source may be separate from the MEMS element 14. The position of the fluid source 20 indicates that gravity may affect the flow within the line-set.

In one preferred embodiment of the system 10, the MEMS element 14 is a MEMS pump 14. As discussed, the MEMS pump 14 in FIG. 1 has an integral power supply. The MEMS pump 14 is capable of pumping fluid contained in the IV bag 20 through the tube 12, out through the access device 24, and into a patient. Once the medication delivery is complete, the system 10 (the tube 12 and MEMS pump 14) may be discarded. It is understood that the IV bag 20 and access device 24 could be considered as parts of the system 10 and may also be disposable.

The medical fluid flow control system 10 is capable of many configurations. Additional elements, including MEMS elements 14, can be added to the system 10. FIG. 2 shows the system 10 with additional elements. Similar elements will be referred to with like reference numerals.

In this form, a MEMS pump 32 is connected to the tubing 12. The MEMS pump 32 has a MEMS local electronics element 36 attached thereto. The MEMS electronics element 36 connects with an external, durable MEMS controller 38. As described in greater detail below, a MEMS flow sensor 30 and a MEMS valve element 34 are also connected to the tubing 12. In a preferred form of the MEMS pump 32, the MEMS electronics element 36 is embedded therein and can preferably store MEMS parametric operational information. The MEMS controller 38, with its electronics and power source, are physically connected to the MEMS electronics element 36. Thus, alternatively, the parametric operational information may be loaded from the detachable MEMS controller 38. In another embodiment, the power source may also originate from the MEMS controller 38. It is understood that the power source could be a MEMS element power source or a power source in other forms known in the art. The MEMS controller 38 may be functionally coupled to the MEMS electronics 36 by a variety of methods including the plug type connection depicted. The system may contain one or multiple electrical connection sites 36 for interface to the durable MEMS controller 38. The MEMS electronics 36 may then be used to locally govern the mechanics of the MEMS pump 32.

The flow sensor 30 can be added to the system 10 to enable more accurate fluid delivery. The flow sensor 30 could also take the form of a pressure sensor if desired. The valve element 34 could alone be added to the typical system to allow metering from a pressurized or otherwise forced system. The flow sensor 30 and valve 34 can assist in controlling the rate of flow and the direction of flow in micro-fluidic circuits and devices in conjunction with the MEMS pump 32 or without the MEMS pump in place.

If desired, the system may also include a slide clamp or other more traditional auxiliary features. A slide clamp may be particularly useful to manually occlude flow in the case of an alarm indicating pump malfunction in a case where the MEMS componentry is normally open. While FIG. 2 shows these various MEMS components to be separate, these MEMS elements could be fabricated as one monolithic unit to be added to the system 10.

The delivery process may implement a normally closed valve 34 or pump 32 designed to open and allow fluid flow only upon sufficient power and appropriate communication transfer to the local electronics element 36 from the controller 38, thereby providing a no-flow condition without the use of cumbersome mechanical devices. This normally closed feature may be integrated directly within other MEMS componentry such as the pump 32 or as a separate MEMS element.

Preferably, the pump element 32 generates the fluid flow through a tube 12 based on information stored locally within the MEMS electronics 36. This information is preferably downloaded from the detachable MEMS controller 38. The direction of fluid flow is preferably from the fluid source 20 into the first tube end 16, directed by the pump 32, through the second tube end 18 to the access device 24 as in medical infusion. In medical infusion configurations, the access device 24 is typically a catheter or needle. The source of fluid in medical infusion devices is generally the IV bag 20 or some type of container. The pump element 32 is instructed by the local MEMS electronics 36 to deliver a controlled amount of medication through the tube 12 to a patient. In the system configuration shown in FIG. 2, the sole reusable element is the controller 38 while the remaining elements are preferably disposable. The controller 38 can control the pump element 32 in a variety of different ways. It can supply intermittent power or power such that the pump element 32 will run in a “slow mode” or a “fast mode.” The controller 38 can supply the power and instructions to the pump element 32 as desired. The reusable controller could be used to download operational information to the MEMS flow control system or could remain docked to the systems throughout the infusion.

Fluid could potentially be directed to flow in the opposite direction. In this embodiment, fluid is drawn by the access device 24, into the second end 18 of the tube 12, due to the action of the pump element 32, with its valves 34 and sensors 30, through the first end 16 of the tube 12, and into the reservoir 20. The medical fluid flow control system 10, in this draw configuration, can be preferably regulated by the use of the pump controller 38 that is electrically connectable to the pump electronics element 36.

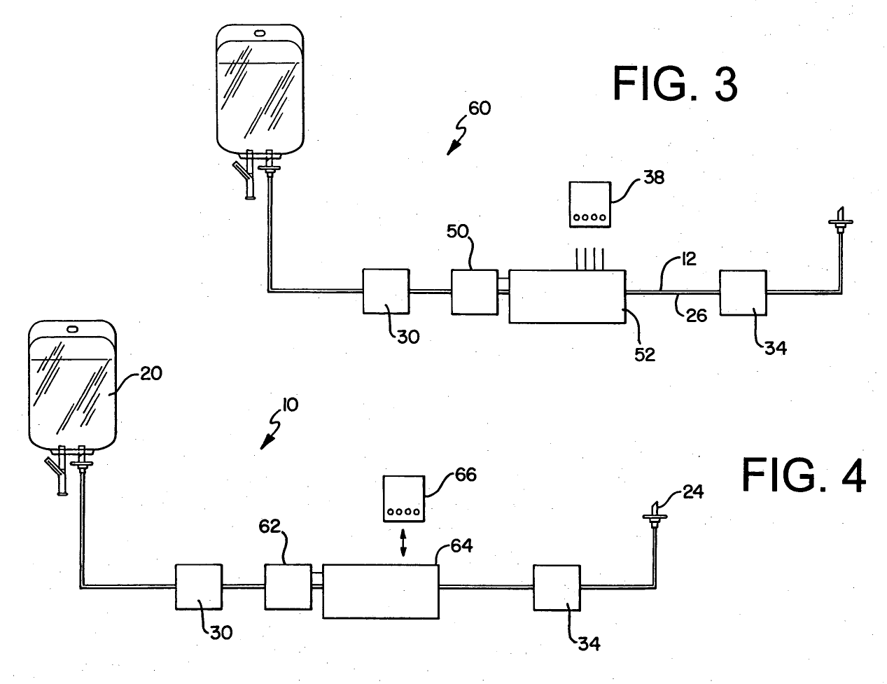

Referring now to FIG. 3, there is shown a diagram of yet another embodiment of the present invention. A power source 50 such as a small battery, fuel cell, or other power supply is added to the system 10 to further decrease the amount of functionality within the durable controller element 38. The power source 50 is preferably connected to the tubing 12 and operably connected to a MEMS pump element 52 similar to the MEMS pump element 32. The power source 50 is designed to last for the life of the MEMS portion of the system. In one embodiment utilizing a fuel cell, the fuel cell 50 is provided as an integral component to an outer surface of the tubing 12. By integral it is meant that the fuel cell 50 is permanently attached to the tubing surface 26 by any suitable means. The power source 50 will also have any necessary activating structure to commence the supply of power. The fuel cell 50 may be any of a myriad of fuel cell designs available and suitable for such use with a line-set such as disclosed in commonly-owned U.S. patent application Ser. No. 10/040,908, Attorney docket number 99-6624 (1417 G P 446) entitled “Medical Infusion System with Integrated Power Supply and Pump Therefore,” the disclosure of which is expressly incorporated herein by reference. While the power supply 50 is shown in FIG. 3 as connected to the MEMS pump 52, it is understood that the power supply 50 could be operably connected to other components as desired.

The use of MEMS or other emerging economical fabrication techniques provide an opportunity to add elements to a disposable line-set for additional functionality such as pumping, valving, and sensing. Some or all of the supporting local electronics could be included in a disposable portion of a line-set as well. For example, it may be preferable to include a memory chip that contains calibration information for a pump 52, pressure sensor and/or flow sensor 30, valve 34, or a combination of disposable elements. Disposability is desirable as it removes the need for costly sterilization or cleaning of the system components between each subsequent application and reduces cost by eliminating a functionality in the durable system componentry.

The durable controller 38 is designed to stimulate fluid distribution quantities directly to the MEMS element 52. This type of controller 38 can be utilized for multiple applications, thus making it reusable. The controller 38 would need minimal alterations for similar reapplication. For example, the dosage for a new patient must be reconfigured by the MEMS element 52 via the reusable controller 38. Such a line-set may in fact be a complete infusion and extrusion system contained in a very small package.

In a preferred embodiment shown in FIG. 3, the MEMS pump element 52 would contain electrical connectivity to enable interface to the durable controller 38 that would control the pump 52 to maintain a desired flow rate. The MEMS pump element 52 can be disposed of with the rest of the disposable components of line-set. The electronics of the controller 38 and any type of case or user's interface would be maintained as a durable, reusable system.

Turning now to FIG. 4, there is pictured a schematic diagram of still another embodiment of the present invention. In this configuration, the system 10 may utilize wireless communication. A MEMS pump 64 is connected to the tube 12. A power supply 62 may be connected to the tube and is operably connected to the pump 64. A wireless controller 66 may be provided to control the MEMS pump 64 or program the related electronics. Wireless communication removes the previous requirement of developing electrical connectivity for the disposable line-set. A wireless linkage will also reduce the complexity of the line-set usage since it will not need to be loaded in as specific a manner as would be the case with hard wired electrical connections. Wireless communication linkage also provides flexibility in terms of usage, for example allowing a disposable, implantable MEMS pump 64 to be controlled by an external system controller 66. It is understood that in a wireless configuration, the MEMS pump 64 will be equipped with appropriate support structure such as to collect energy transmissions and translate power/control to the pump.

In this configuration, the durable, or reusable, wireless controller 66 would communicate via an inductive or capacitive wireless link, with the MEMS pump 64. It is understood that wireless communication could be established with other MEMS components. The MEMS pump 64, or other MEMS components would be disposable but would be provided with the necessary power and electronics to function properly. For example, the disposable elements may require electronics to support the transfer of information from the disposable elements back to the durable controller 66. It is preferable, however, to include as much of the electronics as possible in the durable controller 66 rather than with disposable elements. It may be desirable to maintain sufficient electronics on the disposable side to accept, store, and interpret packets of instruction sets and power so as to reduce required real-time interaction between the durable and disposable portions of the system.

The durable system controller 66 may in turn provide a transfer of information to and from a LAN or other network to fully automate the control and interrogation of the MEMS element 64 into an automated information management system. Optimally, system control and parametric adjustments can be achieved by wireless communication from and to a MEMS system controller 66.

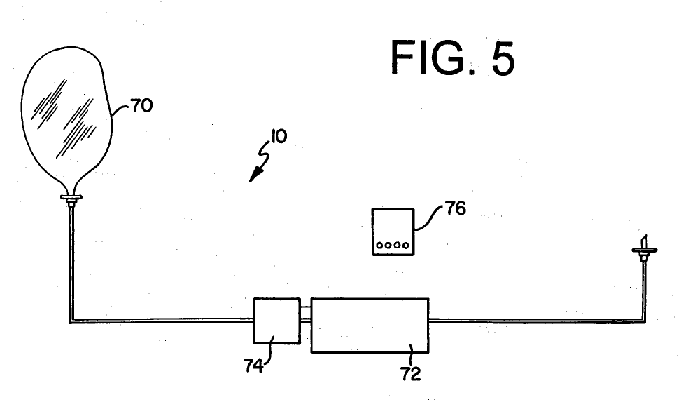

FIG. 5 discloses another embodiment of the medical fluid flow control system 10 of the present invention wherein the system 10 is designed to be implantable within a body. The system 10 utilizes a fluid source or reservoir 70 that is substantially smaller than a conventional IV bag and is disposable. Preferably, a MEMS pump element 72 is connected to the tubing 12. The MEMS pump element 72 has a power supply 74 connected thereto. A wireless controller 76, designed to be remote from the body, communicates wirelessly with the MEMS pump element 72. Thus, all components of the system 10 in FIG. 5 except the controller 76 are designed to be implanted in the body. The durable wireless controller 76 provides the system with the parametric data that the local electronics of the MEMS pump element 72 needs to perform infusion or extrusion.

The fluid reservoir 70 may be refillable and the disposable pieces of the system may include other components such as MEMS valves 34 or sensors 30. Significant advantages over existing methodology include the transfer of mechanical features from a durable system to a disposable portion of the system. This design allows for cheaper construction of the pump controller 76 or durable system 76 and longer-term reliability since the durable system 76 would not include mechanical components. This system also provides the opportunity to develop completely disposable systems or durable/disposable platforms of various fashions.

In another embodiment, the pump 72 itself rather than the reservoir 70 may store and release prescribed amounts of medication into the body. In applications such as an implantable system, there may be no need for an access device 24 in the line-set. A hole or port in the pump 72 may be sufficient to provide a medication exit site from the implanted MEMS system.

The medical fluid flow control system 10 of the present invention may be used when more traditional therapies are considered ineffective or inappropriate. In the case of chronic pain, an infusion and extrusion system is used when oral, intravenous, or topical medications fail to provide effective pain relief or cause uncomfortable side effects. An infusion and draw system can commonly be used when delivering the medication to a specific site or organ is more effective or causes fewer uncomfortable side effects than delivering the medication systemically (to the entire body). The use of a medical fluid flow control system allows a physician to target sites within the body for more effective delivery of a medication. The use of MEMS technology allows more portions of the system 10 to be disposable thus reducing the costs of the system 10. With the use of a MEMS pump having an integral power supply wherein the pump is designed to operate at a single desirable flow-rate, a separate durable controller can be eliminated. Thus, an entire infusion system can be designed from disposable components.

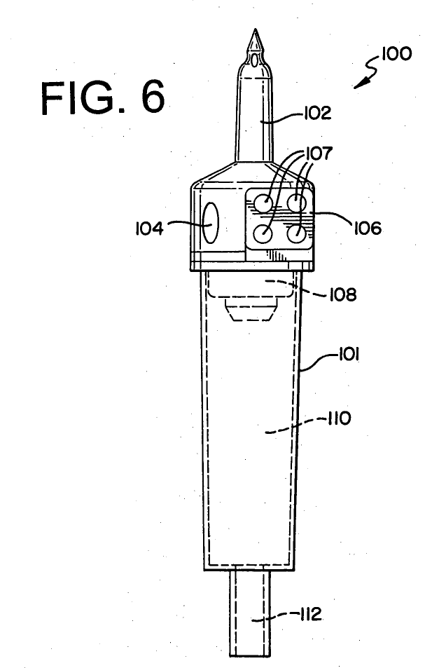

FIG. 6 discloses yet another embodiment of the present invention, a medical spike or spike member 100. In one preferred embodiment, the spike member 100 is a stand-alone member. The stand-alone medical spike 100 can be one disposable component of an infusion system. The spike 100 generally includes a housing 101 defining a passageway 110. The spike 100 further includes a piercing member 102 at one end of the housing 101 and a tube port 112 on the other end of the housing 101. The passageway 110 is situated between the piercing member 102 and the tube port 112. The piercing member 102 is adapted to be inserted into a container, such as an IV bag, vial or similar component. The tube port 112 is adapted to be connected to, for example, a tube, line-set or catheter.

The spike 100 also includes a MEMS element 108 connected to the spike 100. In one preferred embodiment, the MEMS element 108 is housed within the spike 100 proximate the piercing member 102 to facilitate the flow of fluid from the container to the tube, line-set or catheter. Preferably the MEMS element 108 is a MEMS pump. However, the MEMS element 108 may also be a valve, flow sensor, pressure sensor or other similar devices, or a combination thereof. In fact, it may prove useful to load biological or chemical sensors into the spike 100 as a means of assessing infused fluid in a convenient manner and location within the system. The various MEMS items could be fabricated as one unit to be added to the spike 100, or as separate elements connected within the spike 100.

The addition of the flow and pressure sensors with the pump in the MEMS element would enable more accurate delivery of a fluid. The MEMS valve could further facilitate such delivery. Moreover, the valve element would allow metering from a pressurized or otherwise forced system, or from a gravity based system. Additionally, a normally closed valve in such a role could eliminate the need for a slide clamp or roller clamp elsewhere in the system.

The medical spike 100 may require external components to perform or facilitate desired fluid flow control functions. Such external components may include electronics, a power source, a controller and a user interface among others. Several of the external components may operate with the spike 100 through an electrical regulator port 106 connection proximate the MEMS element 108 and piercing member 102. The regulator port 106 shown in FIG. 6 includes four access ports or connection sites 107 to allow for connection to the external components such as a power supply connection (fewer or additional ports 107 can be provided as required for a particular use). An air intake vent 104 can also be integrated into the spike 100 proximate the MEMS element 108 for use in line-set configurations having a rigid reservoir and requiring external air.

The spike 100 can be configured or preprogrammed to provide a single rate of fluid flow, or it may be configured to allow for multiple flow rates. In either event, the rate of flow can be preprogrammed and controlled by the MEMS element 108 (in this instance the MEMS element preferably includes a controller in addition to or instead of a pump).

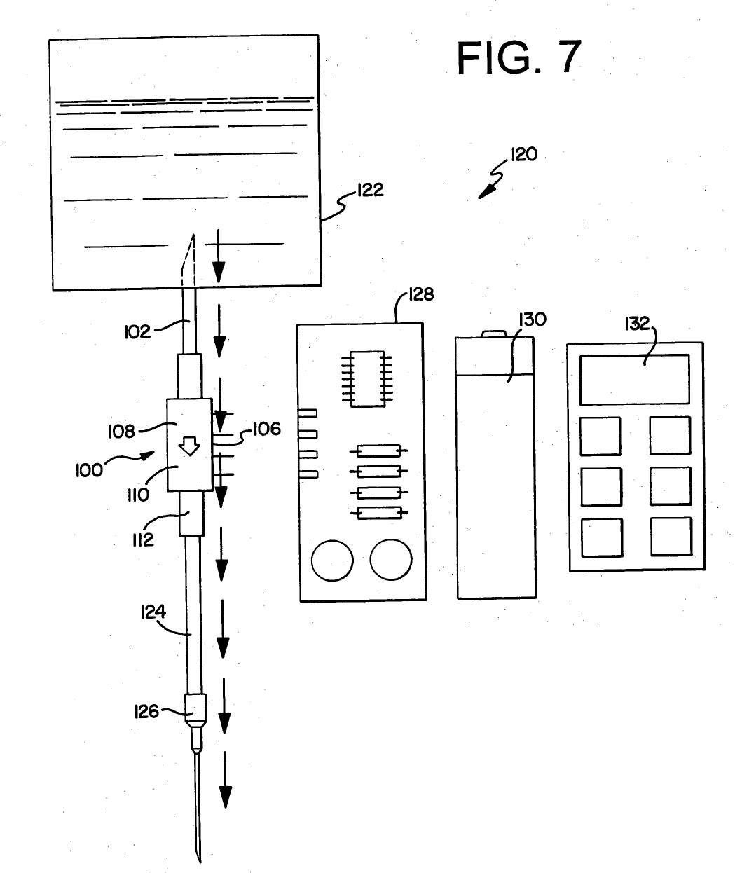

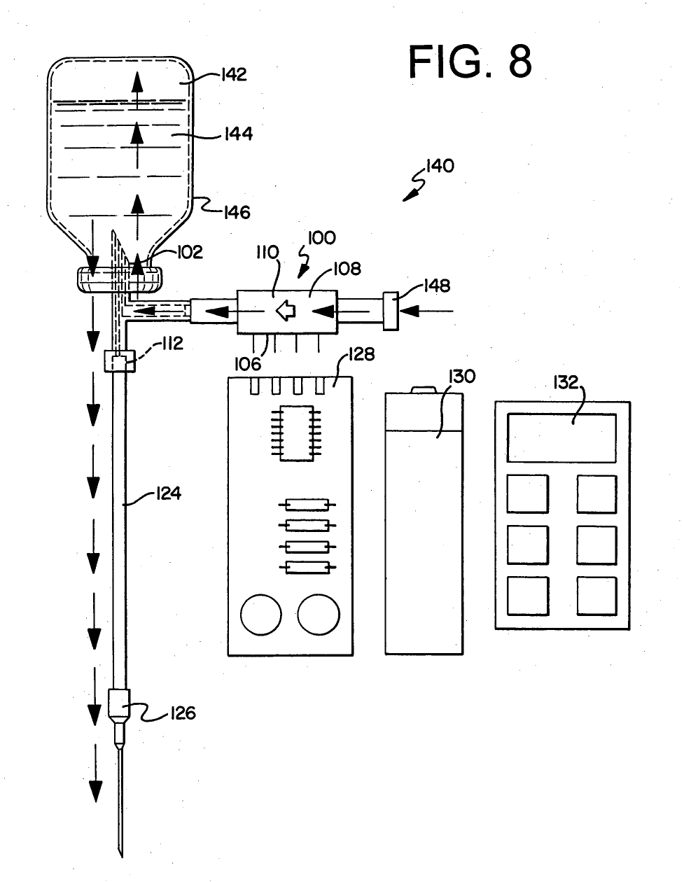

Alternatively, the spike 100 may be a component in an infusion system such as shown in FIG. 7. An external controller 132 can control the rate through an access port in the regulator port 106. The external controller 132 can be utilized to program the spike 100 to provide adjustable flow rates. The controller 132 can also control display of the flow rate, as well as other medical fluid flow control system parametric data, on a display connected to the controller. The external controller 132 may include a hard-wired connection to the spike 100 (through the access port 107) (FIG. 6) or communication may be wireless, by means of a number of viable wireless technologies. Moreover, the controller 132 can be a node in a communication network, permitting the modification of MEMS element parameters from other remote network nodes.

The controller 132 may be a reusable device that also provides user interface features. The controller 132 could be used to program a MEMS controller in the spike 100 and then be removed immediately from the system, or may remain in communication with the spike 100 throughout an infusion session.

The spike 100 can be powered by a battery 130 (see FIG. 7) or other power source, through another of the access ports 107 (FIG. 6) in the regulator port 106. Alternatively, the power source may be integral with the spike 100 and discarded when the entire spike 100 is disposed of. The integral battery may be the sole power supply or may operate in tandem with a more durable power supply on-board or otherwise connected to an external controller. As discussed above, the power supply could also include designs such as disclosed in commonly-owned U.S. patent application Ser. No. 10/040,908, and entitled “Medical Infusion System with Integrated Power Supply and Pump Therefore,” filed on Jan. 7, 2002 and expressly incorporated herein by reference.

The spike 100 can be manufactured to have a traditional or standard external geometry as spikes not having the unique features of the present invention. Alternatively, the external geometry of the spike 100 can be customized to fit with novel reservoir systems.

In an attempt to minimize the durable and reusable components of a medical fluid flow control system using a spike 100, while maximizing the disposable elements, an embodiment of the system 120, as shown in FIG. 7, can include a disposable non-rigid container or reservoir 122 containing a fluid or solution. The reservoir 122 is integrated into the system by piercing a membrane in the non-rigid container 122 with the piercing member 102 of the disposable spike 100. The fluid is drawn from the container 122 by the MEMS element 108 and pumped through a disposable tube 124 to a disposable patient catheter 126. The electronics 128, power source 130, and controller 132, used to monitor and control the infusion system 120, can be removable and reusable. Again, the power source can alternatively be a disposable battery incorporated into the spike 100.

In the configuration shown in FIG. 7, the container 122 collapses as the fluid is pumped from the container 122 by the MEMS element 108 in the spike 100. The flow is monitored and/or controlled by the MEMS element 108 in combination with the external controller 132.

The system can be alternatively configured to fill the reservoir 122 with a fluid. This is accomplished by using the MEMS element 108 of the spike 100 in reverse to pump fluid into the container 122.

Sterilization is of particular concern when medical fluid flow control system components are repeatedly used with different patients. Accordingly, by providing disposable components in the system, this concern is lessened, if not eliminated.

The electronics 128 governing the system 120, the power source 130 for the system and the controller or user interface 132 for controlling and monitoring the system (and in particular, the MEMS element(s) 108) are adapted to connect to the spike through the access ports 107 (FIG. 6) in the regulator port 106. These components can be disconnected from the spike 100 for reuse in controlling and monitoring other disposable infusion systems. Since the electronics 128, power source 130 and controller 132 are not included in the fluid path, they can be reused or durable, although they may need to be cleaned or disinfected as is common practice with infusion pumps.

The spike 100 can also be utilized with a rigid container 146, such as a drug vial, as shown in FIG. 8. In this configuration, the MEMS element 108 can be utilized to pump air 142 (or a variety of other fluids) into the container 146 through an inlet 148. When adding air (or other fluid) to the container 146, the pressure inside the container 146 rises, forcing liquid 144 from the container 146 through the tube port 112 in the spike 100. In this configuration, the liquid 144 from the container 146 does not pass through the passageway 110 in the spike 100. Increasing the pressure increases the flow of liquid 144 through the spike 100 into the patient catheter 126 (or other apparatus connected to the spike 100).

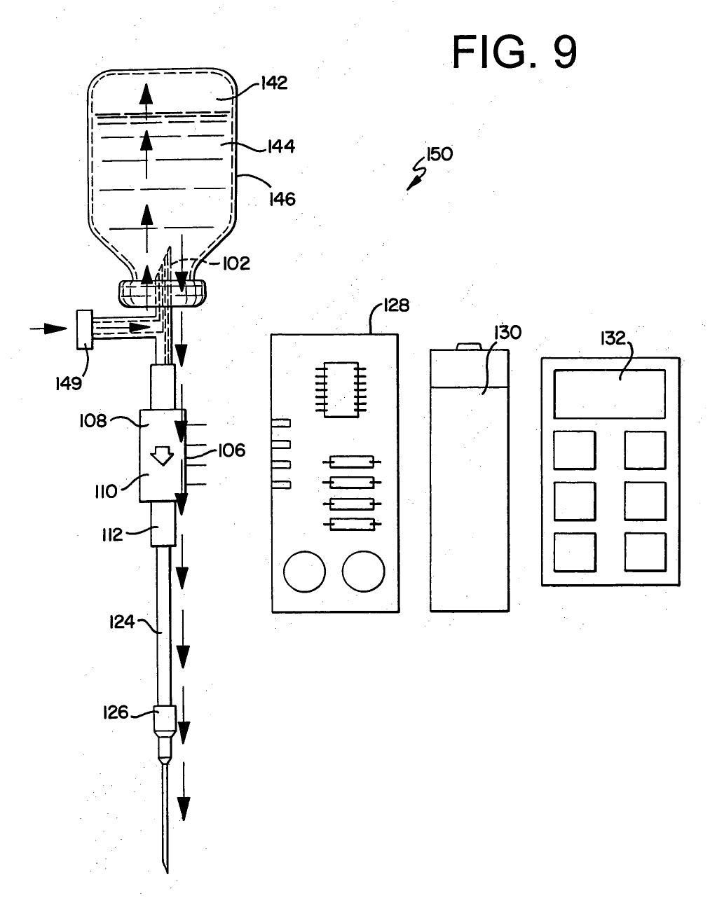

Instead of pumping air (or other fluid) into the rigid container 146, the spike 100 can also be configured to directly pump or draw liquid 144 out of the rigid container 146, as shown in FIG. 9. Unless otherwise acted upon during a pumping operation, the pressure inside the container 146 would be reduced relative to the outside air pressure as the fluid was pumped out of the container 146. In order to normalize the pressure inside and outside the container 146, the spike 100 can include an air intake vent 149. The air intake vent 149 allows the low pressure of the interior of the container 146 to draw air into the container 146 at a rate proportionate to the rate of flow of the outbound fluid. The air intake vent 149 can include a one-way valve (not shown).

Some or all of the supporting electronic elements could be added to the spike or placed at another location in the line-set. For example, it may be preferable to include a memory chip that contains calibration information for a pump or sensor or both, in the spike 100.

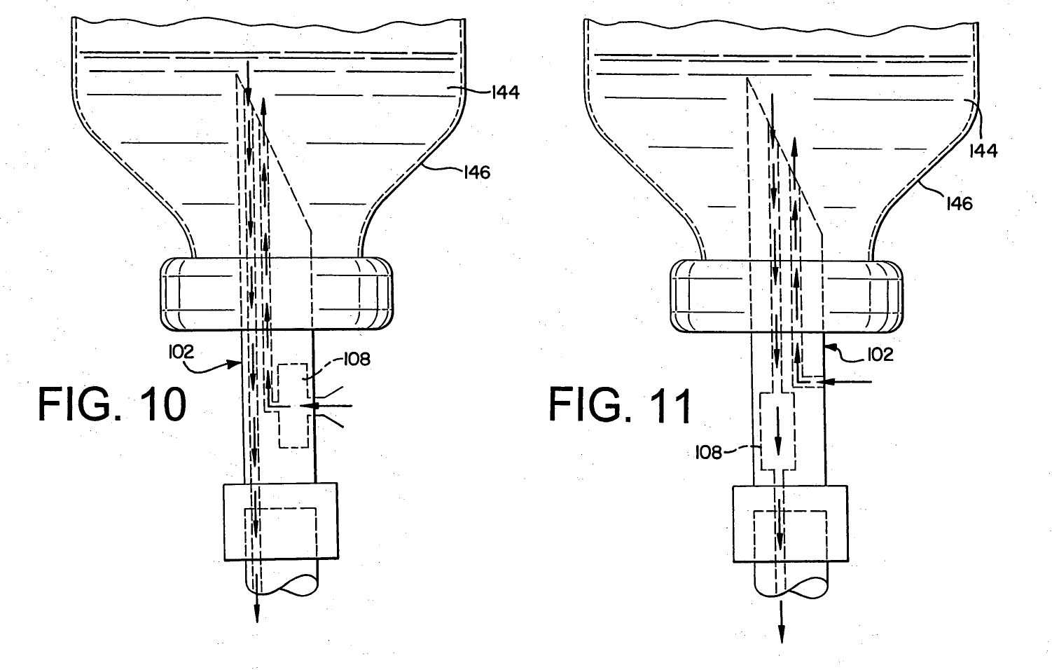

FIGS. 10 and 11 illustrate two additional embodiments where a MEMS pump 108 is housed within a dual-lumen spike 100. FIG. 10 specifically shows a MEMS air pump 108 within the piercing portion 102 of spike 100. FIG. 11 specifically shows the MEMS fluid pump 108 positioned within the piercing portion 102 of spike 100. Similar to the embodiment of FIG. 6, the positioning and type of MEMS component may be varied depending upon the desired operation. The fluid pump is a preferred component for the dual-lumen spike.

Further, operation of these two embodiments is similar to that discussed above for FIGS. 8 and 9, respectively. That is, the fluid pump 108 of FIG. 10 draws air into the pump and discharges the air through a first passageway into the container 146. A high-pressure is created within the container, thereby forcing fluid from the container 146 into the second passageway of the dual-lumen spike 100. Alternatively, as shown in FIG. 11, the fluid pump 108 may draw the fluid from the container 146 creating a low-pressure condition within the container 146. This low-pressure condition draws external fluid (air) through the second passageway to maintain a pressure equilibrium. It may be preferable for all of the supporting electronics, power supply, and memory to be included within the disposable elements of the system. In this scenario, either no reusable controller would exist or the controller would be used to program the pump 108 and then be subsequently removed from the system before use.

Additional features, such as filters or clamps, may be added to the system. A slide clamp may be particularly useful to manually occlude flow in the case of an alarm indicating pump malfunction in the case where the MEMS components are normally open.

While the preferred spike 100 includes a MEMS pump, the spike 100 may also be used (e.g., with a different MEMS element) with an external pump, such as a volumetric pump, an ambulatory pump, a portable or wearable pump, or a gravity based infusion system. For example, it may be preferable to provide flow rate sensors within the spike 100 that communicate to an external infusion pump that is handling the pumping operation.

It is further understood that in any of the embodiments described above, the elements can be configured such that electronics associated with the system are not included with the disposable elements of the system. It is also understood that in a system utilizing a MEMS pump, the pump can run at one preset rate, several discrete rates, or be completely programmable through variation in the controlling electronics. Finally, it is understood that the elements of the several different embodiments described above can be combined or interchanged as is desired.

While the specific embodiments have been illustrated and described, numerous modifications can be made to the present invention, as described, by those of ordinary skill in the art without significantly departing from the spirit of the invention. The breadth of protection afforded this invention should be considered to be limited only by the scope of the accompanying claims.

We claim:

1. A medical infusion system comprising:
a spike member having a fluid passageway; and,
a micro-electromechanical system (MEMS) element operatively connected to the spike member.

2. The system of claim 1 wherein the MEMS element is housed within the spike.

3. The system of claim 1 wherein the spike is disposable.

4. The system of claim 1 further comprising an external controller operatively connected to the spike member for controlling the MEMS element.

5. The system of claim 1 further comprising an external controller operatively connected to the spike member for receiving information from the MEMS element.

6. The system of claim 4 wherein the controller of the MEMS element is wireless.

7. The system of claim 5 wherein the controller of the MEMS element is wireless.

8. The system of claim 4 wherein the controller of the MEMS element is reusable.

9. The system of claim 5 wherein the controller of the MEMS element is reusable.

10. The system of claim 4 wherein the controller displays fluid flow parameters.

11. The system of claim 4 wherein the controller stores fluid flow parameters.

12. The system of claim 4 further comprising a network communication link connectable to the controller.

13. The system of claim 12 wherein the network communication link is capable of transmitting fluid flow parameters to a network of computers.

14. The system of claim 12 wherein the network communication link is capable of controlling the MEMS element remotely.

15. The system of claim 1 further comprising a power source attached to the spike and operably connected to the MEMS element.

16. The system of claim 15 wherein the power source is disposable.

17. The system of claim 1 wherein the MEMS element is selected from the group consisting of a pump, a flow valve, a flow sensor, a pressure sensor and any combination of these elements.

18. The system of claim 1 further comprising a reservoir, wherein the spike is capable of being connected to the reservoir.

19. The system of claim 18 wherein the reservoir comprises a rigid container.

20. A disposable medical line-set comprising:
a length of tubing having a first end;
a fluid extraction spike connected to the first end of the tubing; and
a MEMS pump housed within the fluid extraction spike and operatively connected to the tubing.

21. The disposable medical line-set of claim 20 wherein the spike is configured to attach to a rigid container and comprises an air intake vent member for allowing air into the rigid container proportionate to fluid removed from the rigid container.

22. The disposable medical line-set of claim 21 wherein the MEMS pump draws fluid from the rigid container through the fluid extraction spike.

23. The disposable medical line-set of claim 21 wherein the MEMS pump is configured to force air into the rigid container.

24. The disposable medical line-set of claim 20 further comprising a power source operably connected to the MEMS pump.

25. The disposable medical line-set of claim 20 wherein the spike includes a disposable power source housed within the spike.

26. The disposable medical line-set of claim 20 further comprising a reusable MEMS pump controller communicatively connected to the MEMS pump.

27. The disposable medical line-set of claim 26 wherein the reusable MEMS pump controller is wireless.

28. The disposable medical line-set of claim 20 further comprising a patient catheter connected to a second end of the disposable length of tubing.

29. The disposable medical line-set of claim 28 wherein the patient catheter is disposable.

30. The disposable medical line-set of claim 20 wherein the disposable line-set is capable of being implanted within a body.

31. The disposable medical line-set of claim 20 wherein the spike further comprises a MEMS fluid flow sensor.

32. The disposable medical line-set of claim 20 wherein the spike further comprises a MEMS fluid flow valve.

33. The disposable medical line-set of claim 20 wherein the spike further comprises a MEMS pressure sensor.

34. The disposable medical line-set of claim 26 further comprising a network communication link connectable to the controller.

35. The disposable medical line-set of claim 26 wherein the controller comprises a display for line-set parameters.

36. The disposable medical line-set of claim 26 wherein the controller comprises storage for line-set parameters.

37. A spike member for a medical infusion system comprising:
a housing having a passageway therethrough;
a piercing member connected to one end of the housing and in fluid communication with the passageway; and
a MEMS pump in communication with the passageway and contained within the housing.

38. An infusion system comprising:
a container adapted to contain a flowable substance;
a spike member comprising a passageway therethrough and in fluid communication with the container, the spike having a MEMS pump operatively connected to the spike; and
a system of tubing having one end connected to the spike and in fluid communication with the passageway and another end adapted to be connected to a patient.

39. A medical fluid extraction member comprising:
a substantially rigid body portion having first and second ends;
a first fluid passage having an opening defined at each end of the body and passing therethrough;
a second fluid passage having a first opening defined at the first end of the body and passing a distance through the body and a second opening defined on another portion of the substantially rigid body; and
a MEMS fluid pump operatively communicating with one of either the first fluid passage and the second fluid passage.

40. The medical fluid extraction member of claim 39 further comprising a piercing member at the first end of the body.

41. The medical fluid extraction member of claim 39, wherein the second passage is an air inlet.

42. The medical fluid extraction member of claim 41, wherein the MEMS fluid pump is in operative communication with the air inlet.

43. The medical fluid extraction member of claim 41, wherein the second opening of the second passage is defined on a sidewall portion of the body.

44. The medical fluid extraction member of claim 39, wherein the MEMS fluid pump is in operative communication with the first fluid passage.

45. The medical fluid extraction member of claim 39, wherein the MEMS fluid pump is housed within the substantially rigid body.

46. The medical fluid extraction member of claim 39, wherein the MEMS fluid pump is integral to the substantially rigid body.
